(19) United States
(12) Patent Application Publication
(10) Pub. No.: US 2021/0290947 A1
(43) Pub. Date: Sep. 23, 2021

(54) ARTIFICIAL RETINAL NERVE FLEXIBLE MICROELECTRODE DEVICE AND FABRICATION METHOD THEREOF

(71) Applicant: Wuhan China Star Optoelectronics Technology Co., Ltd., Wuhan (CN)

(72) Inventor: Chengzhi LUO, Wuhan (CN)

(73) Assignee: Wuhan China Star Optoelectronics Technology Co., Ltd., Wuhan (CN)

(21) Appl. No.: 16/625,785

(22) PCT Filed: Nov. 6, 2019
(86) PCT No.: PCT/CN2019/115845

§ 371 Date: Dec. 23, 2019

(30) Foreign Application Priority Data
Jun. 25, 2019 (CN) 201910557024.X

(51) Int. Cl. A61N 1/05; B81C 1/00; A61F 9/00; A61N 1/36; B81B 3/00

(52) U.S. Cl. CPC A61N 1/0543; B81C 1/00349; B81B 3/0097; A61N 1/36046; A61F 9/0017

(57) ABSTRACT

This application discloses an artificial retinal nerve flexible microelectrode device and a fabrication method thereof. The device includes a flexible transparent substrate and a transparent thin film transistor (TFT) array layer. The transparent TFT array layer is disposed on the flexible transparent substrate and used for connecting with an optic nerve in a human eye to transmit a visual electrical signal to the optic nerve.

### FIELD OF INVENTION

This application relates to the field of screenless display technologies, and in particular, to an artificial retinal nerve flexible microelectrode device and a fabrication method thereof.

### BACKGROUND OF INVENTION

Screenless display technology is a method of directly stimulating viewer's visual nerves without a display screen to form specific patterns in the viewer's brain. Conventional artificial retinal nerve flexible array microelectrode chips can only be applied to a blind or verging on blind patient population, and the application range is narrow. When applied to the eyes of a user with certain visual abilities, the device body may block and interfere the user's vision when the device is not being used for display. And currently, there is a lack of a device in the market that can achieve a screenless display function and is widely applicable to patient populations having different visual abilities.

### Technical Problems

The embodiment of the present application provides an artificial retinal nerve flexible microelectrode device and a fabrication method thereof. Each part of the device of the present application is made of a transparent material, so that the whole device is completely transparent. This way, the problem that the existing artificial retinal nerve flexible microelectrode device cannot adapt to users with different visual abilities can be solved.

### Technical Solution

In order to solve the above problems, the technical solution provided by the present application is as follows:

In one aspect, an embodiment of the present application provides an artificial retinal nerve flexible microelectrode device, including:

a flexible transparent substrate;

a transparent thin film transistor (TFT) array layer disposed on the flexible transparent substrate and used for connecting with an optic nerve in a human eye to transmit a visual electrical signal to the optic nerve;

a transparent encapsulation layer disposed on the transparent TFT array layer;

a through hole formed in the transparent encapsulation layer; and

a bridge section extending on the TFT array layer, extending through the through hole, and connected with the optic nerve.

In the artificial retinal nerve flexible microelectrode device provided by the embodiment of the present application, the transparent TFT array layer includes a transparent gate disposed on the flexible transparent substrate, a transparent insulating layer disposed on the flexible transparent substrate and the transparent gate and covering the transparent gate, a transparent active layer disposed on the transparent insulating layer, a transparent source and a transparent drain disposed on the transparent insulating layer and the transparent active layer;

the transparent source and the transparent drain are respectively connected with both ends of the transparent active layer;

the transparent encapsulation layer is disposed on the transparent insulating layer, the transparent active layer, the transparent source, and the transparent drain and covers the transparent active layer and the transparent source;

one end of the bridge section is connected with the transparent drain, and the other end of the bridge section extends through the through hole and connects with the optic nerve.

In the artificial retinal nerve flexible microelectrode device provided by the embodiment of the present application, materials of the flexible transparent substrate and the transparent encapsulation layer are both polyimides.

In the artificial retinal nerve flexible microelectrode device provided by the embodiment of the present application, materials of the transparent gate, the transparent source, the transparent drain and the bridge section are indium tin oxides (ITO).

In the artificial retinal nerve flexible microelectrode device provided by the embodiment of the present application, material of the transparent insulating layer is silicon oxide.

In the artificial retinal nerve flexible microelectrode device provided by the embodiment of the present application, material of the transparent active layer is indium gallium zinc oxide (IGZO).

In the artificial retinal nerve flexible microelectrode device provided by the embodiment of the present application, the flexible transparent substrate has a thickness ranging from 1 um to 5 um, the transparent insulating layer has a thickness ranging from 10 nm to 500 nm, and the transparent active layer has a thickness ranging from 10 nm to 500 nm.

According to the above object of the present application, the application further provides a method for fabricating an artificial retinal nerve flexible microelectrode device, including:

forming a flexible transparent substrate;

forming a transparent TFT array layer used for transmitting a visual electrical signal to the optic nerve on the flexible transparent substrate;

forming a transparent encapsulation layer on the transparent TFT array layer; forming a through hole in the transparent encapsulation layer; and

forming a bridge section extending through the through hole and connected with the optic nerve on the transparent TFT array layer.

In the artificial retinal nerve flexible microelectrode device provided by the embodiment of the present application, the step of forming the flexible transparent substrate includes:

providing a rigid base;

forming the flexible transparent substrate on the rigid base.

In the artificial retinal nerve flexible microelectrode device provided by the embodiment of the present application, the step of forming a transparent TFT array layer used for transmitting a visual electrical signal to the optic nerve on the flexible transparent substrate includes:

forming a transparent gate on the flexible transparent substrate;

forming a transparent insulating layer covering the transparent gate on the flexible transparent substrate and the transparent gate;

forming a transparent active layer on the transparent insulating layer;

forming a transparent source and a transparent drain on the transparent insulating layer and the transparent active layer;

the transparent source and the transparent drain are respectively connected with both ends of the transparent active layer; the transparent encapsulation layer is disposed on the transparent insulating layer, the transparent active layer, the transparent source, and the transparent drain and covers the transparent active layer and the transparent source; one end of the bridge section is connected with the transparent drain, and the other end of the bridge section extends through the through hole and connects with the optic nerve.

### Beneficial Effect

Beneficial effect of the present application: each part of the device is made of a transparent material, so that the whole device is completely transparent, so as to ensure that normal vision of user's eyes is not affected when the device is not being used. Such device has strong applicability to users with different visual abilities. Moreover, the use of the device of the present application achieves the technical effect of screenless display, which not only eliminates manufacturing process of the display screen, but also makes the display more convenient and quicker, saves steps in production process, and makes it suitable for mass production.

### DESCRIPTION OF DRAWINGS

In order to more clearly illustrate the technical solutions in the embodiments or the prior art, the drawings to be used in the embodiments or the prior art will be briefly described below. Obviously, the drawings in the following description are merely some of the embodiments disclosed, and other figures may be obtained based on these drawings by those skilled in the art without any creative work.

FIG. 1 is a schematic structural diagram of an artificial retinal nerve flexible microelectrode device according to an embodiment of the present application.

FIG. 2 is a schematic structural diagram of a transparent TFT array layer in an artificial retinal nerve flexible microelectrode device according to an embodiment of the present application.

FIG. 3 is a schematic block diagram of a method for fabricating an artificial retinal nerve flexible microelectrode device according to an embodiment of the present application.

FIG. 4 is a schematic block diagram of a method for fabricating another artificial retinal nerve flexible microelectrode device according to an embodiment of the present application.

### EMBODIMENTS OF THIS INVENTION

The specific structural and functional details disclosed herein are merely representative and are illustrative of the exemplary embodiments of the present application, but the application may be embodied in many alternative forms and should not be construed as being limited to the embodiments set forth herein.

In the descriptions of the application, it is to be understood that orientation or positional relationships indicated by terms “center”, “transverse”, “upper”, “lower”, “left”, “right”, “vertical”, “horizontal”, “top”, “bottom”, “inner”, “outer”, and the like are orientation or positional relationships shown in the drawings, are adopted not to indicate or imply that indicated devices or components must be in specific orientations or structured and operated in specific orientations, but only to conveniently describe the application and simplify descriptions, so it should not be understood as limits to the application. In addition, terms “first” and “second” are only adopted for description and should not be understood to indicate or imply relative importance or implicitly indicate the number of indicated technical features. Therefore, a feature defined by “first” and “second” may explicitly or implicitly indicate inclusion of one or more such features. In the descriptions of the application, “multiple” means two or more than two, unless otherwise limited definitely and specifically. In addition, the term “comprises” and any variations thereof is intended to cover non-exclusive inclusion.

In the descriptions of the application, it is to be noted that, unless otherwise definitely specified and limited, terms “mount”, “mutually connect” and “connect” should be broadly understood. For example, the terms may refer to fixed connection and may also refer to detachable connection or integrated connection. The terms may refer to mechanical connection and may also refer to electrical connection or mutual communication. The terms may refer to direct mutual connection, may also refer to indirect connection through a medium and may refer to communication in two components or an interaction relationship of the two components. For those of ordinary skill in the art, specific meanings of these terms in the application can be understood according to a specific condition.

The terms used in the description of the present application are only used to describe specific embodiments, and are not intended to show the concepts of the present application. Unless explicitly described to the difference, a singular form includes a plural form in the present specification. In the specification of the present application, it is to be understood that the terms such as “including”, “having”, and “comprising” are intended to indicate the existence of the features, numbers, steps, actions, or combinations in the application, and are not intended to preclude the possibility that one or more other features, numbers, steps, actions, or combinations thereof may exist or may be added.

The present application will be further described below in conjunction with the accompanying drawings and embodiments.

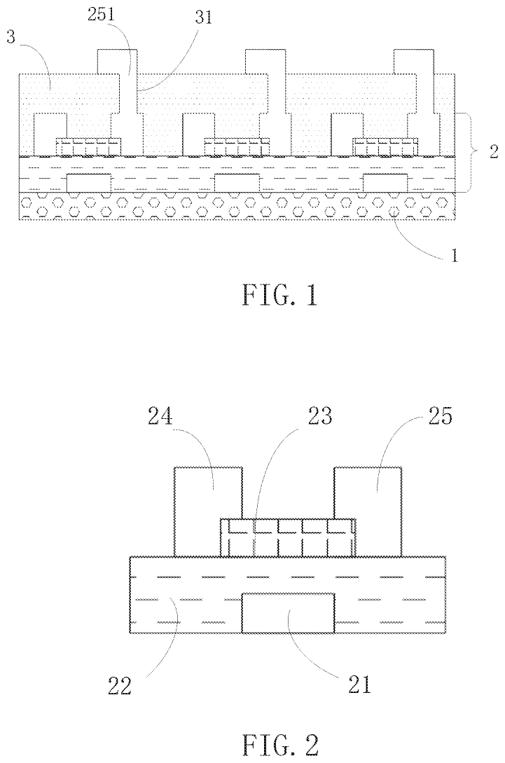

Schematically, as shown in FIG. 1, an embodiment of the present application provides an artificial retinal nerve flexible microelectrode device, including:

a flexible transparent substrate 1; and

a transparent thin film transistor (TFT) array layer 2 disposed on the flexible transparent substrate 1 and used for connecting with an optic nerve in a human eye to transmit a visual electrical signal to the optic nerve.

It can be understood that various types of current visual prostheses all include an array of microelectrodes implanted in eyes for providing electrical stimulation to visual cortex, retina, and optic nerve; specific methods and related technologies for connecting optic nerve in the human eye have also been widely used, which are all prior art, and are not the main problems solved by the beneficial effects of the present application, and will not be further described herein.

The device further includes a transparent encapsulation layer 3 disposed on the transparent TFT array layer 2. Obviously, the transparent encapsulation layer 3 is used to encapsulate the transparent TFT array layer 2. Furthermore, the transparent encapsulation layer 3 also functions as an insulating layer to prevent short-circuiting or crosstalk of the transparent TFT array layer 2 from the optic nerve.

The device further includes a through hole 31 formed in the transparent encapsulation layer 3, and a bridge section 251 extending on the TFT array layer 2, extending through the through hole 31, and connected with the optic nerve. Obviously, a number of bridge sections 251 is the same as a number of thin film transistors arranged in an array in the transparent TFT array layer 2, and the through hole 31 is formed in each of the transparent encapsulation layers 3 corresponding to each of the thin film transistors. Each of the through holes 31 is provided with a bridge section 251 connected to the thin film transistor.

In an embodiment, schematically, as shown in FIG. 2, taking a single thin film transistor as an example, the transparent TFT array layer 2 includes a transparent gate 21 disposed on the flexible transparent substrate 1, a transparent insulating layer 22 disposed on the flexible transparent substrate 1 and the transparent gate 21 and covering the transparent gate 21, a transparent active layer 23 disposed on the transparent insulating layer 22, and a transparent source 24 and a transparent drain 25 disposed on the transparent insulating layer 22 and the transparent active layer 23; the transparent source 24 and the transparent drain 25 are respectively connected with both ends of the transparent active layer 23.

Specifically, the transparent encapsulation layer 3 is disposed on the transparent insulating layer 22, the transparent active layer 23, the transparent source 24, and the transparent drain 25, and covers the transparent active layer 23 and the transparent source 24; the transparent encapsulation layer 3 is combined with the transparent insulating layer 22 to achieve encapsulation of the transparent TFT array layer 2.

Specifically, one end of the bridge section 251 is connected with the transparent drain 25, and the other end of the bridge section 251 extends through the through hole 31 and connects with the optic nerve. It can be understood that the thin film transistor is a functional component commonly used in the field of display, and its working mode and principle are well known to those skilled in the art. In the present application, in particular, the thin film transistor transmits a visual electrical signal to the optic nerve through its transparent drain 25 to complete stimulation of the optic nerve, thereby forming a visual image from the optic nerve to brain, and completing the brain's image acquisition by a screenless display method.

Each component of the flexible microelectrode device of the present application is made of a transparent material, so that the flexible microelectrode device is completely transparent as a whole, and the flexible microelectrode device of the present application embedded in an eyeball has a transparent property, When not in use, it will not interfere with the user's line of sight and will not affect the user's normal visual ability. In an embodiment, specifically, materials of the flexible transparent substrate 1 and the transparent encapsulation layer 3 are both polyimides (PI); materials of the transparent gate 21, the transparent source 24, the transparent drain 25, and the bridge section 251 are indium tin oxides (ITO); material of the transparent insulating layer 22 is silicon oxide (SiOx); and material of the transparent active layer 23 is indium gallium zinc oxide (IGZO). Wherein, the polyimides (PI), indium tin oxides (ITO), silicon oxide (SiOx), and indium gallium zinc oxide (IGZO) have good transparency; in particular, the transparency of silicon oxide (SiOx) is closer to that of glass. Moreover, polyimides (PI) and indium tin oxides (ITO) are respectively used as transparent films and transparent electrodes, which are widely used in the field of optoelectronics.

In an embodiment, the flexible transparent substrate 1 has a thickness ranging from 1 um to 5 um, the transparent insulating layer 22 has a thickness ranging from 10 nm to 500 nm, and the transparent active layer 23 has a thickness ranging from 10 nm to 500 nm. Obviously, less thickness of each functional layer makes the whole device smaller and more refined, and more suitable for implantation into the human eye for replacing or completing related imaging steps or functions.

In summary, each part of the artificial retinal nerve flexible microelectrode device of the present application is made of a transparent material, so that the whole device is completely transparent, so as to ensure that normal vision of the user's eyes is not affected when the device is not being used. The device has strong applicability to users with different visual abilities. Moreover, the use of the device of the present application achieves the technical effect of screenless display, which not only eliminates manufacturing process of the display screen, but also makes the display more convenient and quicker.

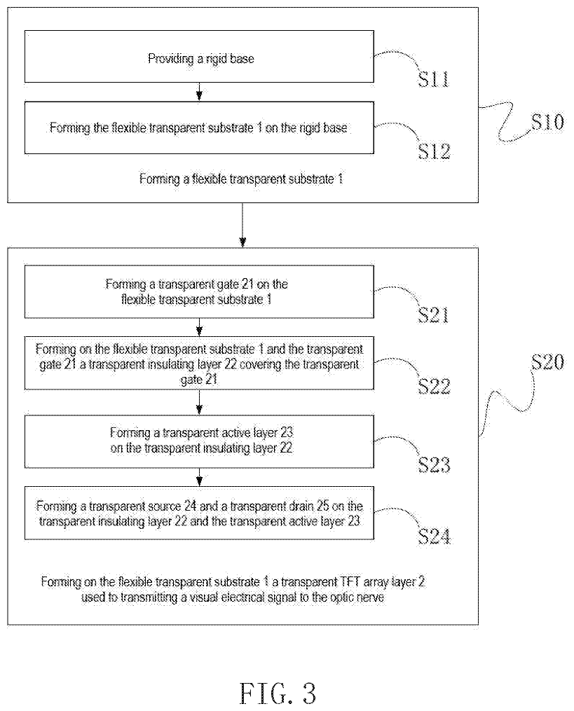

The present application further provides a method of fabricating an artificial retinal nerve flexible microelectrode device, as shown in FIG. 3, including the steps of:

S10: forming a flexible transparent substrate 1.

S20: forming a transparent TFT array layer 2 used for transmitting a visual electrical signal to an optic nerve on the flexible transparent substrate 1.

Specifically, the step S10: forming the flexible transparent substrate 1, including:

S11: providing a rigid base; specifically, the rigid base may be a glass substrate or a silicon plate.

S12: forming the flexible transparent substrate 1 on the rigid base. In an embodiment, the flexible transparent substrate 1 is made of polyimide and has a thickness ranging from 1 um to 5 um; specifically, the flexible transparent substrate 1 may be formed by depositing a uniform layer of polyimide on the rigid substrate.

Specifically, the step S20: forming a transparent TFT array layer 2 used for transmitting the visual electrical signal to the optic nerve on the flexible transparent substrate 1, including:

S21: forming a transparent gate 21 on the flexible transparent substrate 1. In an embodiment, material of the transparent gate 21 is indium tin oxides (ITO); specifically, the transparent gate 21 may be formed by a process such as physical vapor deposition (PVD), exposure, development, and etching.

S22: forming a transparent insulating layer 22 covering the transparent gate 21 on the flexible transparent substrate 1 and the transparent gate 21. In an embodiment, the transparent insulating layer 22 is made of polyimide and has a thickness ranging from 10 nm to 500 nm; specifically, the transparent insulating layer 22 may be formed on the flexible transparent substrate 1 and the transparent gate 21 by means of plasma enhanced chemical vapor deposition (PECVD).

S23: forming a transparent active layer 23 on the transparent insulating layer 22. In an embodiment, the transparent active layer 23 is made of indium gallium zinc oxide and has a thickness ranging from 10 nm to 500 nm; specifically, the transparent active layer 23 having a channel pattern may be formed by a process such as plasma enhanced chemical vapor deposition (PECVD), exposure, development, and etching.

S24: forming a transparent source 24 and a transparent drain 25 on the transparent insulating layer 22 and the transparent active layer 23. In an embodiment, the transparent source 24 and the transparent drain 25 are made of indium tin oxides; specifically, the transparent source 24 and the transparent drain 25 may be formed by processes such as physical vapor deposition (PVD), exposure, development, and etching; wherein the transparent source 24 and the transparent drain 25 are respectively connected with both ends of the transparent active layer 23.

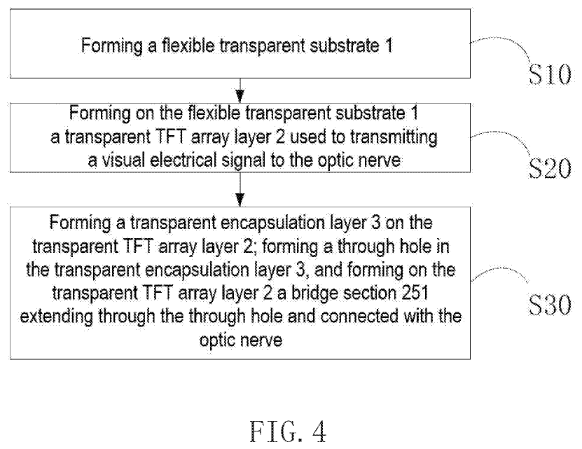

Schematically, as shown in FIG. 4, based on the foregoing fabrication method, the method further includes:

S30: forming a transparent encapsulation layer 3 on the transparent TFT array layer 2, forming a through hole 31 in the transparent encapsulation layer 3, and forming a bridge section 251 extending through the through hole 31 and connecting with the optic nerve on the transparent TFT array layer 2.

In an embodiment, material of the transparent encapsulation layer 3 is polyimide; material of the bridge section 251 is indium tin oxides (ITO); specifically, the transparent encapsulation layer 3 having the through hole 31 may be formed by processes such as deposition, exposure, and development; and then the bridge section 251 may be formed by processes such as physical vapor deposition (PVD), exposure, development, and etching; wherein, specifically, one end of the bridge section 251 is connected with the transparent drain 25, and the other end of the bridge section 251 extends through the through hole 31 and connects with the optic nerve.

Specifically, the transparent encapsulation layer 3 is disposed on the transparent insulating layer 22, the transparent active layer 23, the transparent source 24, and the transparent drain 25, and covers the transparent active layer 23 and the transparent source 24; the transparent encapsulation layer 3 is combined with the transparent insulating layer 22 to achieve encapsulation of the transparent TFT array layer 2.

It should be noted that, after the fabrication methods of FIG. 3 and FIG. 4 are completed, a step of peeling the flexible transparent substrate 1 from the rigid base is further included.

In summary, the present application relates to a method for fabricating an artificial retinal nerve flexible microelectrode device, which realizes mass production of a fully transparent flexible microelectrode device by fabricating various functional components with transparent materials during the fabrication process. At the same time, the fabrication process of each part is relatively mature, and the yield is high. At the same time, the structure of the flexible microelectrode device of the present application is relatively simple, and saves a large number of steps in fabrication process.

In the above, although the present application has been disclosed in the above preferred embodiments, the preferred embodiments are not intended to limit the application. Those skilled in the art can make various modifications without departing from the spirit and scope of the application, and the scope of protection of the present application is determined by the scope defined by the claims.

What is claimed is:

1. An artificial retinal nerve flexible microelectrode device, comprising:
a flexible transparent substrate;
a transparent thin film transistor (TFT) array layer disposed on the flexible transparent substrate and used for connecting with an optic nerve in a human eye to transmit a visual electrical signal to the optic nerve;
a transparent encapsulation layer disposed on the transparent TFT array layer;
a through hole formed in the transparent encapsulation layer; and
a bridge section extending on the TFT array layer, extending through the through hole, and connected with the optic nerve.

2. The artificial retinal nerve flexible microelectrode device as claimed in claim 1, wherein the transparent TFT array layer comprises a transparent gate disposed on the flexible transparent substrate; a transparent insulating layer disposed on the flexible transparent substrate and the transparent gate and covering the transparent gate; a transparent active layer disposed on the transparent insulating layer; a transparent source and a transparent drain disposed on the transparent insulating layer and the transparent active layer;
the transparent source and the transparent drain are respectively connected with both ends of the transparent active layer;
the transparent encapsulation layer is disposed on the transparent insulating layer, the transparent active layer, the transparent source, and the transparent drain, and covers the transparent active layer and the transparent source; and
one end of the bridge section is connected with the transparent drain, and the other end of the bridge section extends through the through hole and connects with the optic nerve.

3. The artificial retinal nerve flexible microelectrode device as claimed in claim 1, wherein materials of the flexible transparent substrate and the transparent encapsulation layer are both polyimides.

4. The artificial retinal nerve flexible microelectrode device as claimed in claim 2, wherein materials of the transparent gate, the transparent source, the transparent drain, and the bridge section are indium tin oxides (ITO).

5. The artificial retinal nerve flexible microelectrode device as claimed in claim 2, wherein material of the transparent insulating layer is silicon oxide.

6. The artificial retinal nerve flexible microelectrode device as claimed in claim 2, wherein material of the transparent active layer is indium gallium zinc oxide (IGZO).

7. The artificial retinal nerve flexible microelectrode device as claimed in claim 2, wherein the flexible transparent substrate has a thickness ranging from 1 um to 5 um, the transparent insulating layer has a thickness ranging from 10 nm to 500 nm, and the transparent active layer has a thickness ranging from 10 nm to 500 nm.

8. A method of fabricating an artificial retinal nerve flexible microelectrode device, comprising the steps of:
forming a flexible transparent substrate;
forming a transparent TFT array layer used for transmitting a visual electrical signal to an optic nerve on the flexible transparent substrate;
forming a transparent encapsulation layer on the transparent TFT array layer; forming a through hole in the transparent encapsulation layer; and
forming a bridge section extending through the through hole and connecting with the optic nerve on the transparent TFT array layer.

9. The method of fabricating the artificial retinal nerve flexible microelectrode device as claimed in claim 8, wherein the step of forming the flexible transparent substrate comprises:
providing a rigid base; and
forming the flexible transparent substrate on the rigid base.

10. The method of fabricating the artificial retinal nerve flexible microelectrode device as claimed in claim 9, wherein the step of forming the transparent TFT array layer used for transmitting the visual electrical signal to the optic nerve on the flexible transparent substrate comprises:
forming a transparent gate on the flexible transparent substrate;
forming a transparent insulating layer covering the transparent gate on the flexible transparent substrate and the transparent gate;
forming a transparent active layer on the transparent insulating layer; and
forming a transparent source and a transparent drain on the transparent insulating layer and the transparent active layer;
wherein the transparent source and the transparent drain are respectively connected with both ends of the transparent active layer; the transparent encapsulation layer is disposed on the transparent insulating layer, the transparent active layer, the transparent source, and the transparent drain, and covers the transparent active layer and the transparent source; and one end of the bridge section is connected with the transparent drain, and the other end of the bridge section extends through the through hole and connects with the optic nerve.
